(19) United States
(12) Plant Patent Application Publication
(10) Pub. No.: US 2015/0096085 P1
(43) Pub. Date: Apr. 2, 2015

(54) Avocado tree named 'Nico'

(50) ;

(71) Applicant: Acosta Farms, Inc., Miami, FL (US)

(72) Inventor: Alcides Acosta, Coral Gables, FL (US)

(73) Assignee: Acosta Farms, Inc., Miami, FL (US)

(21) Appl. No.: 13/998,090

(22) Filed: Sep. 30, 2013

(51) Int. Cl. A01H 5/00

(52) U.S. Cl. PLT/200

(57) ABSTRACT

The ‘Nico’ avocado is of medium size, average 16-22 ounces and being about 5.3 inches in length, and has overall good eating qualities. The fruit matures and can be picked in March to early April. The fruit is an ovate berry that has a soft shelled, smooth textured, exocarp that is dark green.

### LATIN NAME OF THE GENUS AND SPECIES OF THE PLANT AND VARIETY DENOMINATION

Scientific Name: Persea Americana Mille var. ‘Nico’.

### BRIEF SUMMARY OF THE INVENTION

The tree is in a 40 acre avocado grove located at in Miami-Dade County, Fla., at 14395 Southwest 200th Street, Miami, Fla. 33187. The tree is estimated to be between 20 to 25 years old and has been in production for at least 15 years. The fruit differ from other avocados because it ripens later than others. Its origin is unknown. Its fruit can be stored at temperatures above 50° Fahrenheit.

Parentage is unknown as the tree was discovered, and is of a wild nature. The tree has been observed for 20 to 25 years, and has been in production for 15 years. A comparison of the microsatellite patter from this avocado against 13 known varieties (Lula, Simmonds, Monroe, Choquette, Semil-34, Semil-43, Melendez, Hall, Booth & Hardee, April, Carla, and Pollock) revealed no matches.

Asexual propagation was attained in 1998 in Miami-Dade County, Fla. when the plant was 5-10 years old. Asexual reproduction was achieved by grafting cuttings of the tree onto new avocado seedlings. The tree differs from other late varieties on the lateness of the maturity of the fruit. ‘Nico’ presents resistance to carpospores purpurea pathogens.

### BRIEF DESCRIPTION OF THE DRAWINGS

FIG. 1 shows the trunk at a lower portion of the Nico avocado tree.

FIG. 2 shows the midlevel portion of the Nico avocado tree.

FIG. 3 shows the upper portion of the Nico avocado tree.

FIG. 4 shows a detailed view of the leaf of the Nico avocado tree near the base.

FIG. 5 shows the scale in inches of a typical fruit of the Nico avocado tree measured against a ruler.

FIG. 6 shows the color and internal configuration of the fruit and the seed of the Nico avocado tree.

### DETAILED BOTANICAL DESCRIPTION

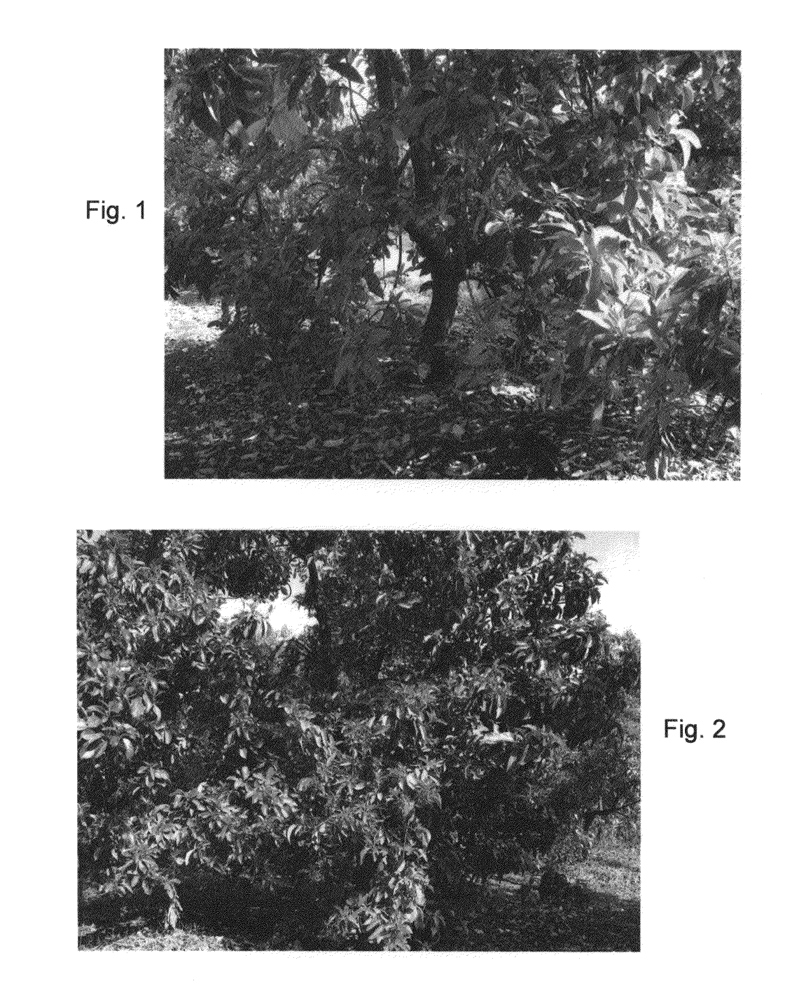

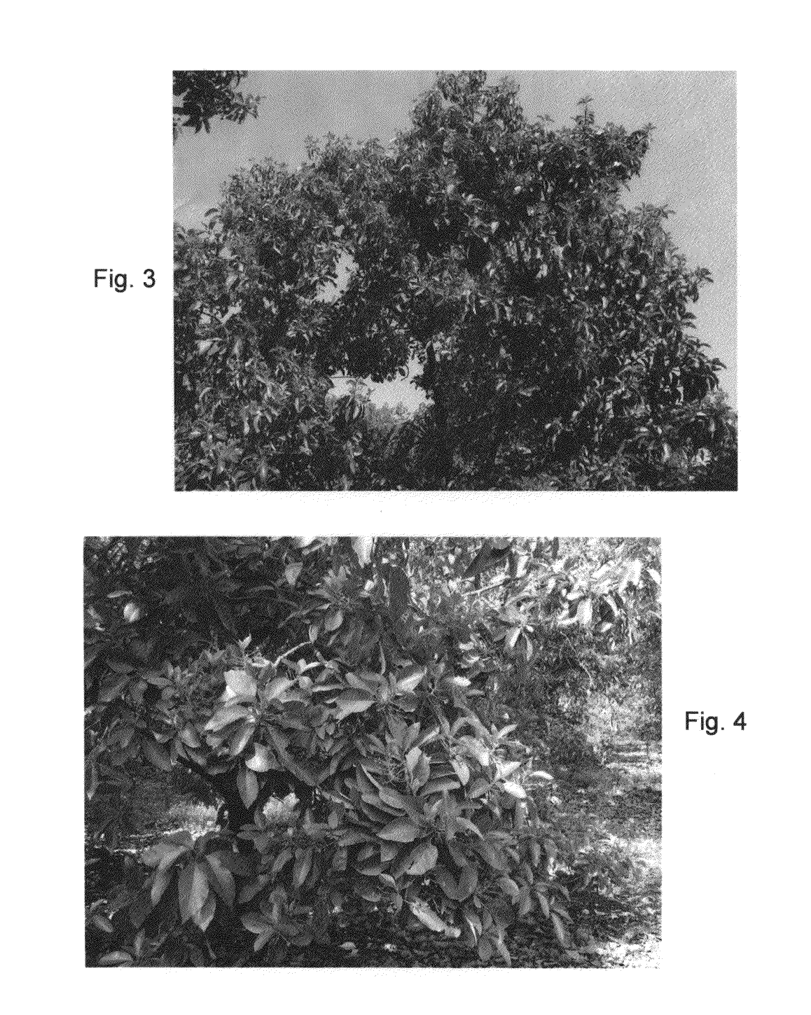

The tree presents a vigorous upright growth with a spread of 15′ and 25′ high, as shown in FIGS. 1, 2, 3 and 4. As shown in FIG. 1, the diameter of the main trunk is 22″ tapering as it extends straight and upward to the pinnacle of the tree. The main trunk splits into 4 branches each about 3½ inches in width with the vertical sub trunk splitting into smaller branches. The bark of the trunk is light in color with new shoots being green in color and the foliage being vivid green.

The leaves are dark green, 2½″ to 3″ wide by 9″ in length, smooth surface becoming a leathery texture as they mature, as appears in FIGS. 1, 2, 3, and 4. The foliage is of heavy density near the ground, becoming sparse toward the upper regions of the tree. Typical observed petiole diameter is 3.4-3.8 mm. Buds are yellow-green, have an average length of 8-9 mm, average width of 3.5 mm, and an oblong shape with a rounded base and acute tip. Pedicels are green-yellow in color and average 5-6 mm in length and 1.2 mm in width with an average diameter of 1 mm. Peduncles range from 41 mm to 151 mm and average about 85 mm and are yellow green in color. Typical observed flower depth is 7.5-8 mm.

The flowers of this plant have 3 lobes that are approximately 6.5 mm in length and 2 mm in width. Margins are smooth and uniform, and the texture is smooth. Densely silk tomentose is found on both surfaces. The shape is apex acute with a flattened base. Flowers typically have three sepals of about 6 mm in length and 3 mm in width. Margins are smooth and uniform, smooth in texture, and having densely silk tomentose on both surfaces. The shape is apex acute with a flattened base. Fruit shape is ovate, averaging 4″ in diameter.

The seed is round, weighing about 6.9 oz, depending on overall fruit size, having a diameter between 2.5 and 3″, and fitting in a tight cavity. The plant produces fruit at a medium-high volume, averaging about 275-300 lbs per year.

The tree's winter hardness is 9B. Avocado shelf life is several weeks, and the fruit can be stored above 50°. The fruit naturally ripens once placed at room temperature or about 5-8 days after harvesting.

The flowers are not fragrant. The tree blooms in March and the blooms are of the B type. Fruit matures and can be picked in March or early April of the following year. The fruit weights are between 16 and 22 ounces with the diameter averaging 4 inches. The fruit is full in flavor and peels easily. The seed is tight in the cavity and is 3 inches long and dark brown in color.

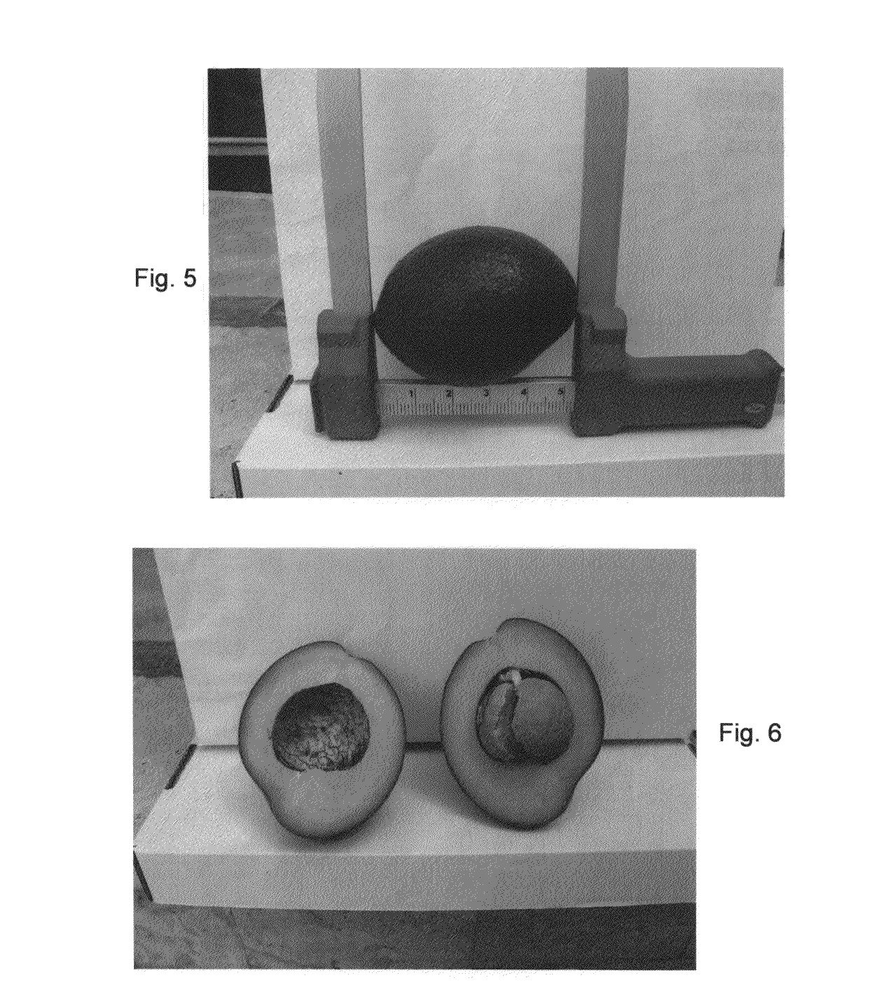

FIG. 5 shows the outside of the fruit, which is an ovate berry that matures in March. It has a thin ( 1/32 inch) soft shelled exocarp, which is smooth textured, and dark green. The fruit weighs 12-16 ounces and averages 5.3 inches in length, as shown in FIG. 5.

In FIG. 6, the interior of the fruit is seen to have a mesocarp color that is green near the shell and yellow near the seed cavity.

1. A new and distinct variety of avocado tree substantially as described and illustrated and characterized as to novelty by its overall good eating qualities, its medium size and its being ready to be picked in March to early April.
